(19) United States
(12) Patent Application Publication
(10) Pub. No.: US 2025/0186225 A1
(43) Pub. Date: Jun. 12, 2025

(54) Robotic Cement Pressurizer

(71) Applicant: Howmedica Osteonics Corp., Mahwah, NJ (US)

(72) Inventors: Christine Perrone Ehlers, Warwick, NY (US); Mark Ellsworth Nadzadi, Memphis, TN (US)

(21) Appl. No.: 18/535,246

(22) Filed: Dec. 11, 2023

(51) Int. Cl. A61F 2/46

(52) U.S. Cl. CPC A61F 2/461; A61F 2/4657; A61F 2002/4631; A61F 2002/4632; A61F 2002/469

(57) ABSTRACT

Disclosed herein are systems and methods for securing orthopedic implants using a robotic system. The robotic system can include a surgical robotic manipulator, an end effector coupled to the manipulator, and a controller configured to cause the end effector to hold an implant in a final implant position within a patient's anatomy until the controller determines that cement located adjacent to the implant has reached a predetermined cure state. A method of placing an orthopedic implant can include preparing a bone to receive an implant in a final implant position within the bone, including applying cement to a cavity in the bone, and employing a robotic surgical system to execute an automated process of using an end effector to hold the implant in the cavity until the cement has reached a predetermined cure state.

### FIELD OF INVENTION

The present disclosure relates to systems and methods for securing orthopedic implants, and particularly to systems and methods for securing orthopedic implants using a robotic cement pressurizer.

### BACKGROUND OF THE INVENTION

Bone cement can act as a stabilizing adhesive for orthopedic implants to provide a secure and stable environment that is essential for proper tissue and bone healing. By providing a strong bond between the implant and the bone, bone cement prevents the implant from migrating and provides a secure hold. Cementing technique has a direct impact on the success of a cemented joint replacement implant system. For example, during a typical total or partial knee arthroplasty, a surgeon may insert a tibial insert trial that is slight larger than the final insert size, which allows for the cement to be pushed deeper into the cancellous bone. While this practice is beneficial to provide an optimal strong cement to bone to implant bond and prevent implant micromotion, too much tension can destabilize the surrounding soft tissue and lead to an increase in aseptic loosening of the implant leading to revision surgery.

Furthermore, cementing technique is generally performed manually by surgeons, and the details of the technique can vary significantly between them. This may lead to inaccurate or unreliable results.

Therefore, there exists a need for proper cementing techniques to promote long-term implant stability and reduce the likelihood of implant loosening.

### BRIEF SUMMARY OF THE INVENTION

Disclosed herein are systems and methods for securing orthopedic implants with cement using a robotic system.

In accordance with an aspect of the present disclosure, a robotic system is provided. A robotic system according to this aspect, may include a surgical robotic manipulator, an end effector coupled to the manipulator, and a controller configured to cause the end effector to hold an implant in a final implant position within a patient's anatomy until the controller determines that cement located adjacent to the implant has reached a predetermined cure state.

Continuing in accordance with this aspect, the predetermined cure state may be a completely cured state.

Continuing in accordance with this aspect, the controller may be configured to cause the end effector to move in synchrony with a portion of the patient's anatomy within which the final implant position may be located while holding the implant in the final implant position. The robotic system may include a localizer configured to monitor a position of the portion of the patient's anatomy and transmit signals representative of the position to the controller.

Continuing in accordance with this aspect, the controller may be configured to cause the end effector to press against the implant with a predetermined force while holding the implant in the final position.

Continuing in accordance with this aspect, the determination that cement located adjacent to the implant has reached the predetermined cure state may be based upon passage of a predetermined amount of time while the end effector holds the implant in the final position.

Continuing in accordance with this aspect, the controller may be configured to automatically release the implant from the end effector when the controller determines that the cement located adjacent to the implant has cured.

Continuing in accordance with this aspect, the controller may be configured to cause the end effector to lock in place such that the end effector may be prevented from moving and maintains the implant in the final position until the controller determines that cement located adjacent to the implant has reached the predetermined cure state.

Continuing in accordance with this aspect, the controller may be configured to use the end effector to press the implant along an insertion path to a depth to achieve a desired thickness of the cement between the implant and the patient's anatomy.

Continuing in accordance with this aspect, the robotic system may include a positioning tool provided on the end effector. The positioning tool may be configured to receive an implant specifically for knee replacement surgery.

In accordance with another aspect of the present disclosure, a method of placing an orthopedic implant is provided. A method according to this aspect may include the steps of preparing a bone to receive an implant in a final implant position within the bone, including applying cement to a cavity in the bone, and employing a robotic surgical system to execute an automated process of using an end effector to hold the implant in the cavity until the cement has reached a predetermined cure state.

Continuing in accordance with this aspect, the implant may be an implant for knee replacement surgery.

Continuing in accordance with this aspect, the predetermined cure state may be a completely cured state.

Continuing in accordance with this aspect, the automated process may include controlling the end effector to move in synchrony with a portion of a patient's anatomy within which the final implant position may be located while holding the implant in the final implant position.

Continuing in accordance with this aspect, the automated process may include controlling the end effector to press against the implant with a predetermined force while holding the implant in the final position.

Continuing in accordance with this aspect, the automated process may include an automated determination of whether the cement has reached the predetermined cure state. The automated determination may be based upon passage of a predetermined amount of time while the end effector holds the implant in the final position.

Continuing in accordance with this aspect, the automated process may include automatically releasing the implant from the end effector when a controller determines that the cement located adjacent to the implant has cured.

Continuing in accordance with this aspect, the automated process may include locking the end effector in place such that the end effector may be prevented from moving and maintains the implant in the final position until a controller determines that cement located adjacent to the implant has reached the predetermined cure state.

Continuing in accordance with this aspect, the automated process may include using the end effector to press the implant along an insertion path to a depth to achieve a desired thickness of the cement between the implant and the bone.

### BRIEF DESCRIPTION OF THE DRAWINGS

A more complete appreciation of the subject matter of the present disclosure and the various advantages thereof can be realized by reference to the following detailed description, in which reference is made to the following accompanying drawings:

FIG. 1 is perspective view of a robotic system according to an embodiment of the present disclosure;

FIG. 2 is a perspective view of an end effector of the robotic system of FIG. 1;

FIG. 3 is a side view of the end effector of FIG. 2;

FIG. 4 is a schematic view of a display screen of the robotic system of FIG. 1, and

FIGS. 5A-5C shows various steps of a robotic cementing procedure according to an embodiment of the present disclosure.

### DETAILED DESCRIPTION

Reference will now be made in detail to the various embodiments of the present disclosure illustrated in the accompanying drawings. The term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “cement” and “bone cement” will be used interchangeably and as such, unless otherwise stated, the explicit use of either term is inclusive of the other term. Similarly, the terms “implant” and “prosthesis” will be used interchangeably and as such, unless otherwise stated, the explicit use of either term is inclusive of the other term.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior” means closer to the head, and the term “inferior” means more distant from the head.

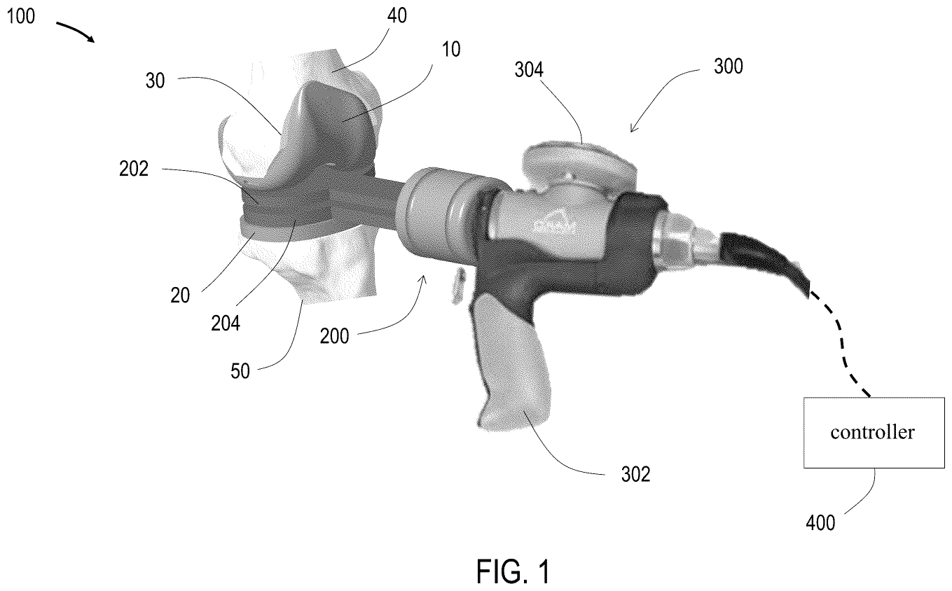

Described below is a robotic system 100 for securing orthopedic implants with cement. Robotic system 100 can include an end effector 200 coupled to a surgical robotic manipulator 300. A controller 400 of the robotic system can control movement, speed, end effector functions, etc. As shown in FIG. 1, robotic system 100 can be used to secure implants during a knee arthroplasty according to an embodiment of the present disclosure. According to this embodiment, end effector 200 is an adjustable and expandable spacer that functions as a robotic cement pressurizer to secure a femoral implant 10 to a femur 40 and a tibial implant 20 to a tibia 50 with cement 30. Cement 30 can include any bone cement such as cements based on polymethyl methacrylate (PMMA). Robotic cement pressurizer 200 allows for accurate and consistent implant fixation using cement. Controller 400 can be programmed to provide precise, consistent cement pressure and curing duration by controlling the speed and force of application in both the loading and unloading phases, helping to maximize cement quality and minimize impaction and misapplication. Robotic cement pressurizer 200 allows for a high degree of precision and repeatability, increasing accuracy while minimizing risks and complications of over tensioning the surrounding soft tissue.

The manipulator of robotic system 100 can be a handpiece 300 as shown in FIG. 1. Handpiece 300 is configured to readily couple and uncouple with the robotic cement pressurizer. Various functions of robotic cement pressurizer can be controlled by controller 400 via handpiece 300. For example, by adjusting the distance between a first paddle 202 and a second paddle 204 of robotic cement pressurizer 200, controller 400 can alter the distraction height and consequently the pressure applied to the femoral and tibial implant during the cement curing and fixation. By controlling the distraction height—the distance between the two paddles-controller 400 is able to modify the amount and duration of pressure applied, allowing an operator to customize cement fixation based on the characteristics and needs of the patient. Handpiece 300 includes a grip 302 to allow an operator to conveniently grip the handpiece and manipulate the attached robotic cement pressurizer 200. An interface 304 allows handpiece 300 to be attached to a robotic arm (not shown) making up robotic system 100.

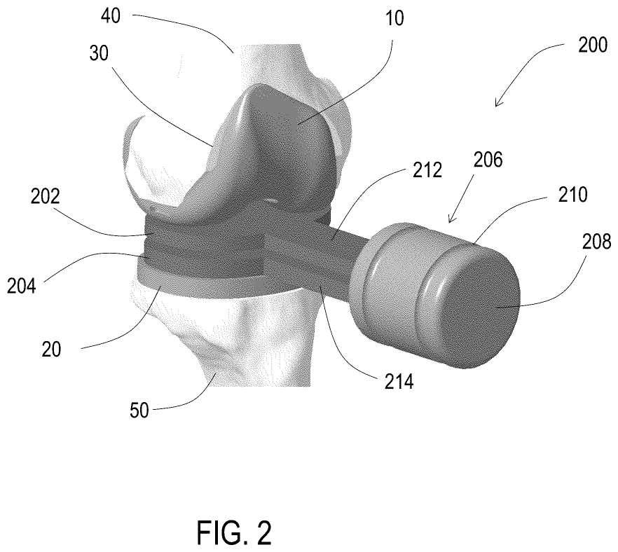

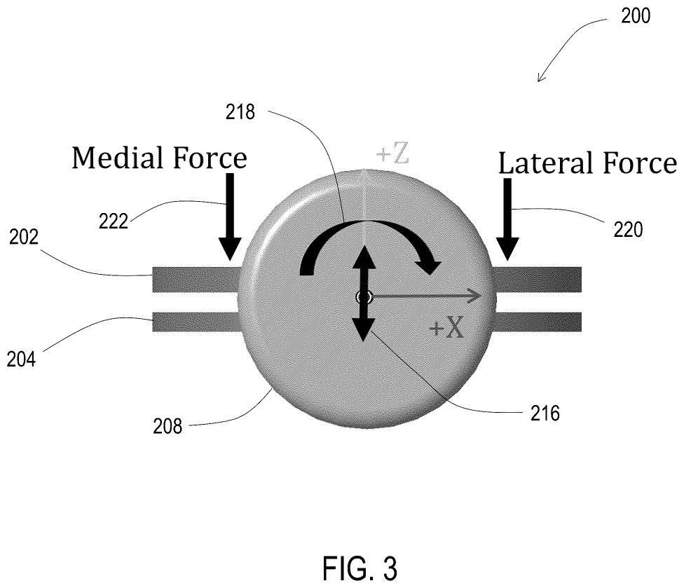

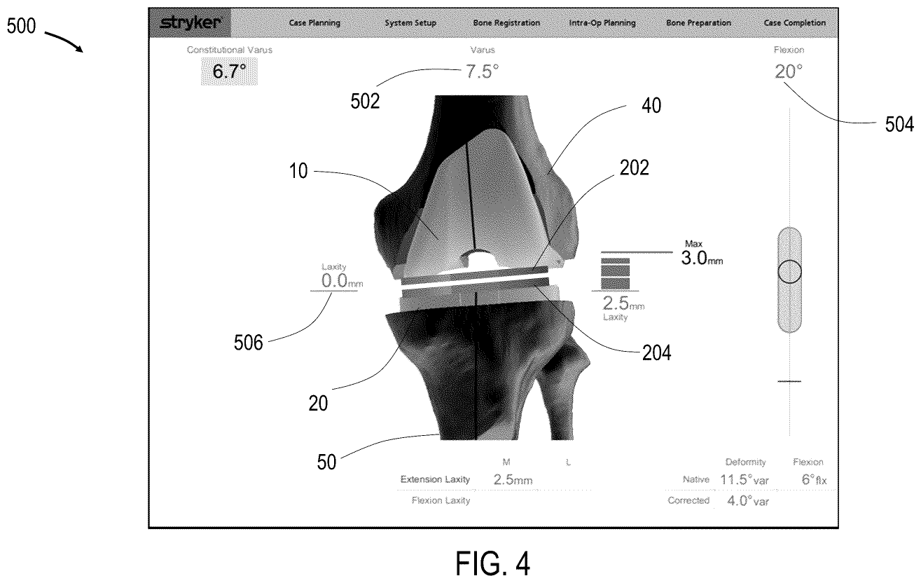

FIGS. 2 and 3 show details of robotic cement pressurizer 200, which functions as a mechanical device that applies pressure to both femoral implant 10 and tibial implant 20 (tibial baseplate) of a patient's knee joint during the cement curing process. First paddle 202 is a femoral paddle, designed to contact the lateral and medial sides of femoral implant 10. Moving first paddle 202 toward femoral implant 10 will push the femoral implant against the curing cement 30 and the femur 40, thereby applying controlled pressure to the femoral implant while the cement cures. Similarly, second paddle 204 is a tibial paddle, designed to contact the tibial implant 20. By pushing the second paddle toward tibial implant 20, the implant will press against curing cement 30 and the tibia 50, thereby subjecting the tibial implant to an appropriate pressure during cement curing. Both first and second paddles are connected to a connector 206 situated in the robotic cement pressurizer. The surface of the paddles can be made of a soft material such as rubber, foam, silicon, or other elastic material that will ensure that the contact is not damaging to the implants but with sufficient rigidity. This can include any material designed to prevent any scratching or abrasive contact with the implants. A first arm 212 and a second arm 214 (as best seen in FIG. 2) attach the connector to the two paddles, thereby providing the necessary connection. By manipulation of the first and second paddles via connector 206, a distraction distance between the two paddles can be controlled precisely. Thus, the distance between the first and second paddles can be changed depending on the level of pressure applied to the femoral and tibial implants. Increasing the distance between the two will decrease the amount of pressure applied, and vice versa. The pressure applied to the femoral implant can be specifically adjusted to match that of the tibial implant, or it can be made different from it instead. For example, the robotic system can apply an additional upward force on handpiece 300 toward the femoral component, augmenting the pressure levels on the femoral component with respect to the tibial component.

Connector 206 has a generally cylindrical body in this embodiment with a circular face 208 to allow for quick connection to handpiece 300. The cylindrical body of connector 206 is designed to ensure a snug fit so that the robotic cement pressurizer 200 functions optimally and securely when connected to handpiece 300. Various features on connector 206 such as grooves 210 allow for ready and secure reversible connection to handpiece 300 as best shown in FIG. 2. Thus, robotic cement pressurizer 200 can be easily connected to apply pressure and be just as easily and readily disconnected to instantaneously stop or gradually reduce the application of pressure to the implants.

As shown in FIG. 3, robotic cement pressurizer 200 can perform various functions to securely and precisely attach the femoral and tibial implants. Moving the first and second paddles apart along the Z-axis can increase or decrease a distraction distance 216 which in turn controls the amount of pressure being applied to the implants. Rotating connector 206 along the Y-axis will change a varus-valgus 218 of the patient's knee joint and impact the balance between a medial force 222 applied on the medial side and a lateral force 220 applied on the lateral side of the robotic cement pressurizer. These features allow the operator and/or controller 400 to make precise, secure adjustments to the patient's knee joint depending on the desired outcome of the surgery. One or more force sensors on robotic cement pressurizer 200 can be used to measure forces on both sides of the femoral and tibial implants. The force sensors can be located on the first and second paddles or elsewhere on robotic cement pressurizer 200. One or more torque sensors on robotic cement pressurizer 200 can measure the torque applied for varus-valgus adjustments.

Referring now to FIG. 3, a display screen 500 of robotic system 100 according to an embodiment of the present disclosure is shown. Display screen 500 displays in real time the patient's knee joint, the implants, and the robotic cement pressurizer 200 during a surgical procedure. It also displays changes in the orientation or position of the robotic cement pressurizer in order to demonstrate the impact this has on implant placement and the cement curing process. For example, varus-valgus 502 values are shown to allow the surgeon to set the desired value for the specific patient by adjusting the rotation of the first and second paddles of robotic cement pressurizer 200. A joint range of motion 504 is also displayed to indicate the position of the knee joint in flexion and extension. This position can be determined by a localizer or camera (not shown) that records and transmits the knee joint position to controller 400 when the knee joint is moved between flexion and extension. Using this information, controller 400 can ensure that the robotic cement pressurizer is positioned and moved accordingly in order to apply the desired pressure to the implants. Also shown on display screen 500 are laxity values 506 in various flexion and extension positions for the medial and lateral sides, allowing the surgeon to adjust distraction levels to the desired levels.

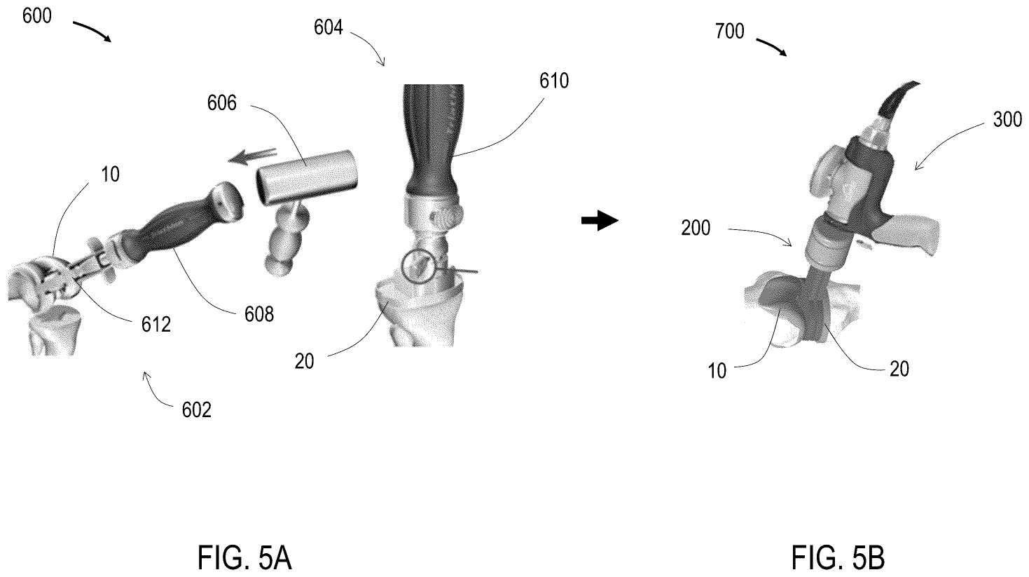

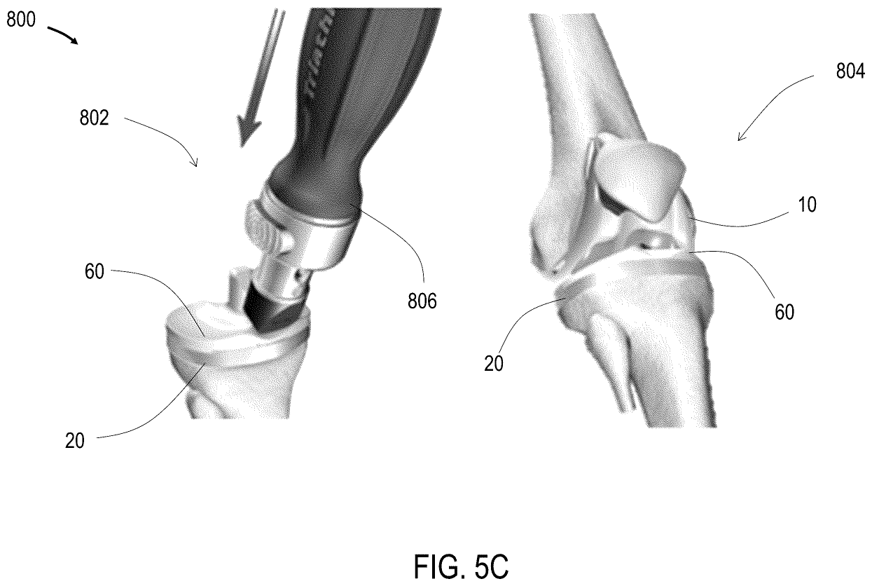

FIGS. 5A-5C show various steps of a robotic cementing procedure using robotic system 100 according to an embodiment of the present disclosure. A first step 600 includes a femoral implant placement step 602 and a tibial implant placement step 604 as shown in FIG. 5A. Once the bone beds of the distal femur and the proximal tibia are prepared for cement application, sticky cement is applied to the implant and/or bone beds prior to the implant placement steps. The quantity of cement, type of cement, cement temperature, cement viscosity, application locations, etc. can be provided by controller 400 of robotic system 100. For example, an operator can enter patient specifics such as patient demographics, type of surgical procedure, implant type, etc., into controller 400. Once this data is entered, controller 400 will output a set of instructions on using the cement in the surgery, such as the amount of cement to apply, curation duration, cured state detection, and the force needed to secure the implant in place. After application of cement 30, femoral implant 10 is attached to femoral impactor/extractor 612. An impactor 606 and a femoral impaction handle 608 is used to firmly position femoral implant 10 to the resected femur 40. Similarly, tibial implant is attached to a tibial impaction handle 610 and attached to the resected tibia 50 as shown in FIG. 5A.

Robotic cement pressurizer 200 of robotic system 100 is used in a cement curing step 700 to ensure precise and accurate implant placement as shown in FIG. 5B and more fully explained above. By utilizing controller 400, an operator can accurately control the pressure and duration of the cement curing cycle with unmatched precision. This enables the operator to deliver consistent or varying cement pressure and duration of application while loading and unloading during the implanting process, allowing for maximum cement quality while minimizing any risk of impaction or misapplication. The highly precise and repeatable nature of the robotic cement pressurizer 200 allows surgeons to accurately and safely implant an implant with minimal disruption to the surrounding tissue and maximum effectiveness of the cement. In one embodiment, controller 400 can be programmed to calculate and target the proper pressure to be applied to implants during the cement curing step in order to ensure optimal implant fixation—i.e., when a predetermined cure state is achieved. The predetermined cure state and the pressure required to achieve same can be established through a study involving experimental pressurizing of femoral and tibial components on cadaver bone or sawbones specimens, followed by micromotion testing and sectioning to measure the depth of cement penetration in the bone. Following analysis of the results, the optimal pressure over time can be determined. In this way, an optimal amount of pressure is reached which avoids over tensioning of the surrounding soft tissue.

During the cement curing step, load or force sensors of robotic system 100 can measure the load feedback from the application of pressure onto the tibial and femoral paddles and adjust accordingly to ensure that a consistent pressure is present until the bone cement is hardened. Torque sensors of robotic system 100 allow for adjustments to be made in varus/valgus and distraction for the medial/lateral compartments in order to ensure that a final poly insert or tibial insert implant 60 is implanted correctly in a step 802 using an impaction handle 806 as shown in FIG. 5C. This calculated and precise application and duration of pressure during the cement curing process provides ideal linkage between the implant and the cement as well as the cement and the implant itself resulting long term implant stability.

While the disclosure herein generally discusses embodiments directed to a knee joint, the systems and methods disclosed here can be used for any other joint such as a hip, shoulder, ankle, etc. Further, the methods and systems disclosed herein are not limited to joint implants, but can be used to secure any implant with cement.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. A robotic system, comprising:
a surgical robotic manipulator;
an end effector coupled to the manipulator; and
a controller configured to:
cause the end effector to hold an implant in a final implant position within a patient's anatomy until the controller determines that cement located adjacent to the implant has reached a predetermined cure state.

2. The robotic system of claim 1, wherein the predetermined cure state is a completely cured state.

3. The robotic system of claim 1, wherein the controller is configured to cause the end effector to move in synchrony with a portion of the patient's anatomy within which the final implant position is located while holding the implant in the final implant position.

4. The robotic system of claim 3, comprising a localizer configured to monitor a position of the portion of the patient's anatomy and transmit signals representative of the position to the controller.

5. The robotic system of claim 1, wherein the controller is configured to cause the end effector to press against the implant with a predetermined force while holding the implant in the final position.

6. The robotic system of claim 1, wherein the determination that cement located adjacent to the implant has reached the predetermined cure state is based upon passage of a predetermined amount of time while the end effector holds the implant in the final position.

7. The robotic system of claim 1, wherein the controller is configured to automatically release the implant from the end effector when the controller determines that the cement located adjacent to the implant has cured.

8. The robotic system of claim 1, wherein the controller is configured to cause the end effector to lock in place such that the end effector is prevented from moving and maintains the implant in the final position until the controller determines that cement located adjacent to the implant has reached the predetermined cure state.

9. The robotic system of claim 1, wherein the controller is configured to use the end effector to press the implant along an insertion path to a depth to achieve a desired thickness of the cement between the implant and the patient's anatomy.

10. The robotic system of claim 1, comprising a positioning tool provided on the end effector, wherein the positioning tool is configured to receive an implant specifically for knee replacement surgery.

11. A method of placing an orthopedic implant, the method comprising:
preparing a bone to receive an implant in a final implant position within the bone, including applying cement to a cavity in the bone; and
employing a robotic surgical system to execute an automated process of using an end effector to hold the implant in the cavity until the cement has reached a predetermined cure state.

12. The method of claim 11, wherein the implant is an implant for knee replacement surgery.

13. The method of claim 11, wherein the predetermined cure state may be a completely cured state.

14. The method of claim 11, wherein the automated process includes controlling the end effector to move in synchrony with a portion of a patient's anatomy within which the final implant position is located while holding the implant in the final implant position.

15. The method of claim 11, wherein the automated process includes controlling the end effector to press against the implant with a predetermined force while holding the implant in the final position.

16. The method of claim 11, wherein the automated process includes an automated determination of whether the cement has reached the predetermined cure state.

17. The method of claim 16, wherein the automated determination is based upon passage of a predetermined amount of time while the end effector holds the implant in the final position.

18. The method of claim 11, wherein the automated process includes automatically releasing the implant from the end effector when a controller determines that the cement located adjacent to the implant has cured.

19. The method of claim 11, wherein the automated process includes locking the end effector in place such that the end effector is prevented from moving and maintains the implant in the final position until a controller determines that cement located adjacent to the implant has reached the predetermined cure state.

20. The method of claim 11, wherein the automated process includes using the end effector to press the implant along an insertion path to a depth to achieve a desired thickness of the cement between the implant and the bone.
